(19) United States
(12) Patent Application Publication
(10) Pub. No.: US 2020/0107742 A1
(43) Pub. Date: Apr. 9, 2020

(54) ELECTROENCEPHALOGRAM MEASURING APPARATUS

(71) Applicant: NIHON KOHDEN CORPORATION, Tokyo (JP)

(72) Inventors: Michinori NAKAGAWA, Tokorozawa-shi (JP); Ryuzo MASE, Tokorozawa-shi (JP)

(21) Appl. No.: 16/585,043

(22) Filed: Sep. 27, 2019

(30) Foreign Application Priority Data
Sep. 28, 2018 (JP) 2018-184190
Sep. 10, 2019 (JP) 2019-164326

(51) Int. Cl. A61B 5/0478; A61B 5/00

(52) U.S. Cl. CPC A61B 5/0478; A61B 5/7415; A61B 2562/04; A61B 2560/0418; A61B 2562/0209; A61B 5/6806

(57) ABSTRACT

An electroencephalogram measuring apparatus includes: a first electrode that is to be placed in a first position of a head of a subject; a second electrode that is to be placed in a second position of the subject; and a body supporting at least the first electrode, and having a portion that is to be held by a hand of a user.

### CROSS-REFERENCE TO RELATED APPLICATIONS

This application is based upon and claims the benefit of priority from Japanese Patent Application No. 2018-184190 filed on Sep. 28, 2018 and Japanese Patent Application No. 2019-164326 filed on Sep. 10, 2019.

### TECHNICAL FIELD

The present disclosure relates an electroencephalogram measuring apparatus.

### BACKGROUND

An electroencephalogram is an electrical signal which is generated by the brain activity of a human being. An electroencephalogram measuring apparatus measures an electroencephalogram by using electroencephalogram electrodes that are attached to the scalp of the subject. The measured electroencephalogram is varied depending on a position in the scalp. When an electroencephalogram is to be measured, therefore, a number of electroencephalogram electrodes must be placed in respective predetermined positions of the scalp. The International 10-20 electrode placement system shows examples of electroencephalogram electrode placement.

In an electroencephalogram measuring apparatus disclosed in U.S. Pat. No. 5,293,867, many electroencephalogram electrodes are supported on a head cap that is to be mounted on the head of the subject. The placement of the electroencephalogram electrodes is determined in accordance with the International 10-20 electrode placement system.

Recently, a diagnosis based on an electroencephalogram measurement is expected also in the field of emergency medicine. In the case of the electroencephalogram measuring apparatus disclosed in U.S. Pat. No. 5,293,867, however, the placement of the many electroencephalogram electrodes in the all predetermined positions requires a long time. Therefore, there is a possibility that the apparatus cannot respond to an emergency situation.

The present disclosure provides an electroencephalogram measuring apparatus that assists more rapid and easier acquisition of an electroencephalogram.

### SUMMARY

According to an aspect of the present disclosure, there is provided an electroencephalogram measuring apparatus comprising: a first electrode that is to be placed in a first position of a head of a subject; a second electrode that is to be placed in a second position of the subject; and a body supporting at least the first electrode, and having a portion that is to be held by a hand of a user.

### BRIEF DESCRIPTION OF DRAWINGS

FIG. 1 illustrates the functional configuration of an electroencephalogram measuring apparatus that is common to embodiments.

FIG. 2 illustrates an electroencephalogram measuring apparatus of a first embodiment.

FIG. 3 illustrates an electroencephalogram measuring apparatus of a second embodiment.

FIG. 4 illustrates a modification of the electroencephalogram measuring apparatus of FIG. 3.

FIGS. 5A and 5B illustrate modifications of the electroencephalogram measuring apparatus of FIG. 3.

FIG. 6 illustrates an electroencephalogram measuring apparatus of a third embodiment.

FIG. 7 illustrates an electroencephalogram measuring apparatus of a fourth embodiment.

### DESCRIPTION OF EMBODIMENTS

Hereinafter, embodiments of the present disclosure will be described in detail with reference to the drawings. In the drawings, in order to make components of a described object recognizable, the scales of the components to be described are adequately changed.

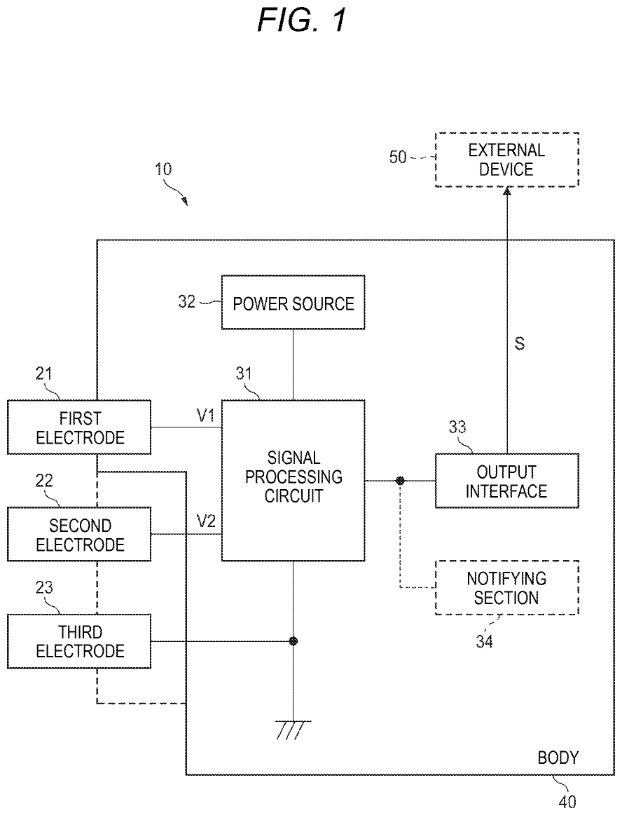

FIG. 1 illustrates the functional configuration of an electroencephalogram measuring apparatus 10 that is common to the embodiments. The electroencephalogram measuring apparatus 10 may include a first electrode 21, a second electrode 22, a third electrode 23, a signal processing circuit 31, a power source 32, and an output interface 33.

The first electrode 21 is electrically conductive. The first electrode 21 is placed in a portion which is in the head of the subject, and from which an electroencephalogram is to be measured, and detects a first potential V1 of the portion.

The second electrode 22 is electrically conductive. The second electrode 22 is placed in a portion of the body of the subject to detect a second potential V2 of the portion. The portion of the body in which the second electrode 22 is to be placed will be specifically described later.

The first electrode 21 and the second electrode 22 are electrically connected to the signal processing circuit 31. The first potential V1 that is detected by the first electrode 21, and the second potential V2 that is detected by the second electrode 22 are supplied to the signal processing circuit 31.

The signal processing circuit 31 is configured so as to acquire the potential difference between the first potential V1 and the second potential V2. A change over time of the potential difference corresponds to the electroencephalogram of the subject. The signal processing circuit 31 may appropriately include an amplifying circuit and filtering circuit that are used for obtaining a desired output signal. The power source 32 supplies an electric power for enabling the signal processing circuit 31 to execute predetermined processes.

The third electrode 23 is electrically conductive. The third electrode 23 is placed in an arbitrary portion, and provides a potential that functions as the reference of the potential difference between the first potential V1 and the second potential V2.

In the following description, the signal that is output from the signal processing circuit 31 is referred to as the electroencephalogram signal S. The electroencephalogram signal S is supplied to an external device 50 through the output interface 33. Examples of the external device 50 are an electroencephalograph and a patient monitor. The connection between the output interface 33 and the external device 50 may be realized by a wired connection, or configured so that a wireless connection is enabled.

The electroencephalogram measuring apparatus 10 may include a body 40. The first electrode 21 is supported by the body 40. The signal processing circuit 31, the power source 32, and the output interface 33 are housed in the body 40. In this case, the power source 32 may be a primary battery or a secondary battery. Alternatively, at least one of the signal processing circuit 31, the power source 32 and the output interface 33 may be placed outside the body 40. In the case where the power source 32 is placed outside the body 40, a commercial power supply or a generator may be used as the power source 32.

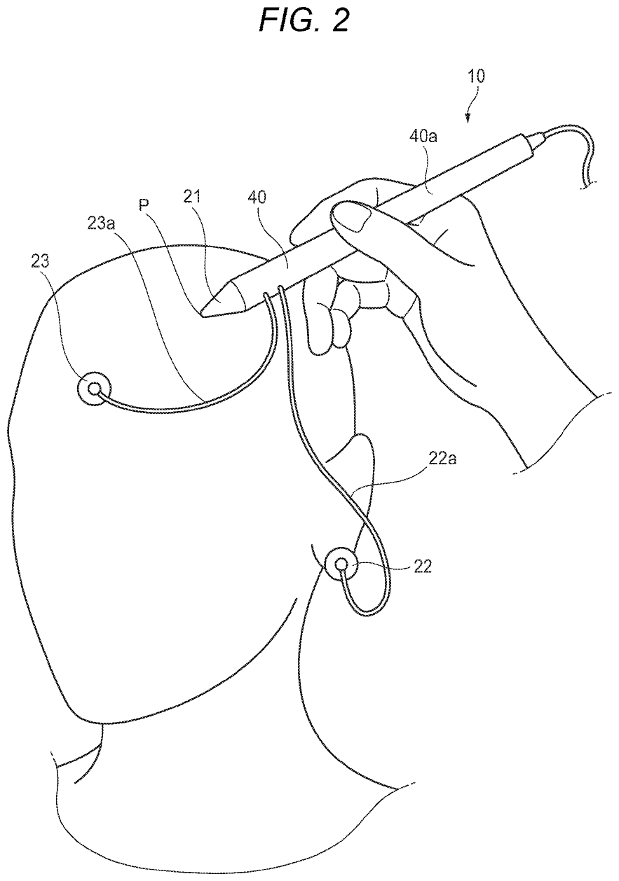

FIG. 2 illustrates a first embodiment of the electroencephalogram measuring apparatus 10. In the embodiment, the body 40 exhibits a pen-like appearance. The body 40 may include a holder 40a that is to be held by the hand of the user during measurement.

The first electrode 21 is supported on the tip end of the pen-like body 40. The second electrode 22 is connected to the body 40 through a signal line 22a. The third electrode 23 is connected to the body 40 through a signal line 23a.

The second electrode 22 is attached to, for example, the ear lobe of the subject. The third electrode 23 is attached to a portion that is less movable, and that is stable, such as the forehead. The user presses the first electrode 21 against a position P which is in the head, and from which an electroencephalogram is to be measured. The illustrated position P is an example of the first position. The ear lobe is an example of the second position.

According to the configuration, an electroencephalogram at the position P is obtained. A signal indicating the electroencephalogram (the electroencephalogram signal S in FIG. 1) is output through the output interface 33.

In an electroencephalogram measurement method according to the International 10-20 electrode placement system, the work of attaching many electroencephalogram electrodes to the all predetermined portions requires a long time. In a field where rapid recognition of an abnormality of the brain of the patient is requested, such as an emergency medical care, it is required to acquire an electroencephalogram more rapidly and easily.

According to the above-described configuration, an electroencephalogram of the subject corresponding to the potential difference between the first electrode 21 and the second electrode 22 can be acquired by performing a simple operation in which the second electrode 22 and the third electrode 23 are attached to appropriate positions, and then the first electrode 21 is placed in the arbitrary position P of the head of the subject. Even during measurement, moreover, the position where the first electrode 21 is placed can be freely changed with the user's hand that holds the holder 40a. Therefore, it is possible to assist more rapid and easier acquisition of an electroencephalogram that is requested particularly in a site such as emergency medicine.

In the body 40 in the embodiment, the first electrode 21 is disposed in the tip end portion that is thinner than the holder 40a. In the body 40, alternatively, a configuration where the first electrode 21 is disposed in a tip end portion that is wider than the holder 40a may be employed as in a probe of an ultrasonic diagnosing apparatus.

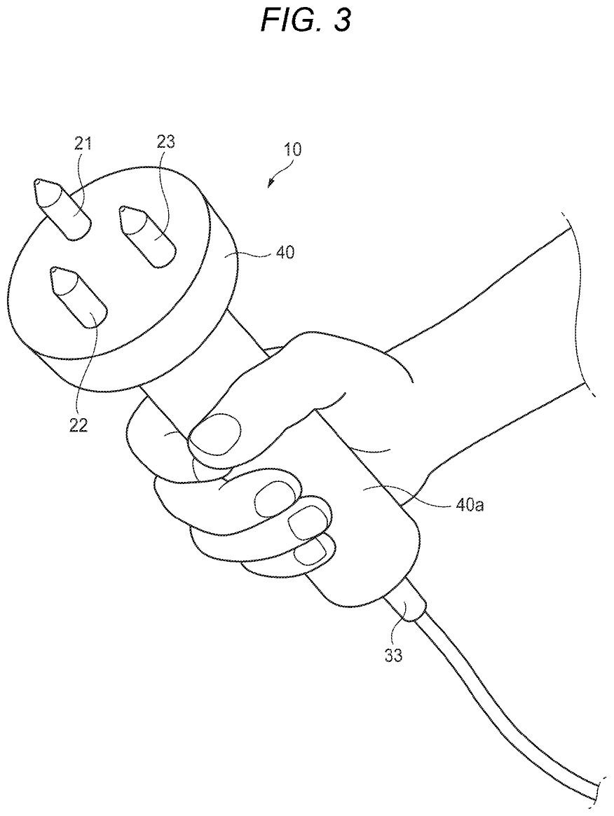

FIG. 3 illustrates a second embodiment of the electroencephalogram measuring apparatus 10 having such a tip end portion. In the embodiment, the first electrode 21, the second electrode 22, and the third electrode 23 are supported by the body 40. The body 40 may include the holder 40a that has a rod-like shape, and that is to be held by the hand of the user during measurement.

The user presses the first electrode 21 against a position which is in the head of the subject, and from which an electroencephalogram is to be measured. Also the second electrode 22 and the third electrode 23 are pressed against the head of the subject. Therefore, an electroencephalogram corresponding to the potential difference between the first electrode 21 and the second electrode 22 is obtained. A signal indicating the electroencephalogram (the electroencephalogram signal S in FIG. 1) is output through the output interface 33.

According to the configuration, the user can acquire an electroencephalogram corresponding to the potential difference between the first electrode 21 and the second electrode 22, by performing a simple operation in which it is required only to, while holding the holder 40a by the hand, press the first electrode 21, second electrode 22, and third electrode 23 that are supported by the body 40, against the head of the subject. Even during measurement, moreover, the position where the first electrode 21 is placed can be freely changed with the user's hand that holds the holder 40a. Therefore, it is possible to assist more rapid and easier acquisition of an electroencephalogram.

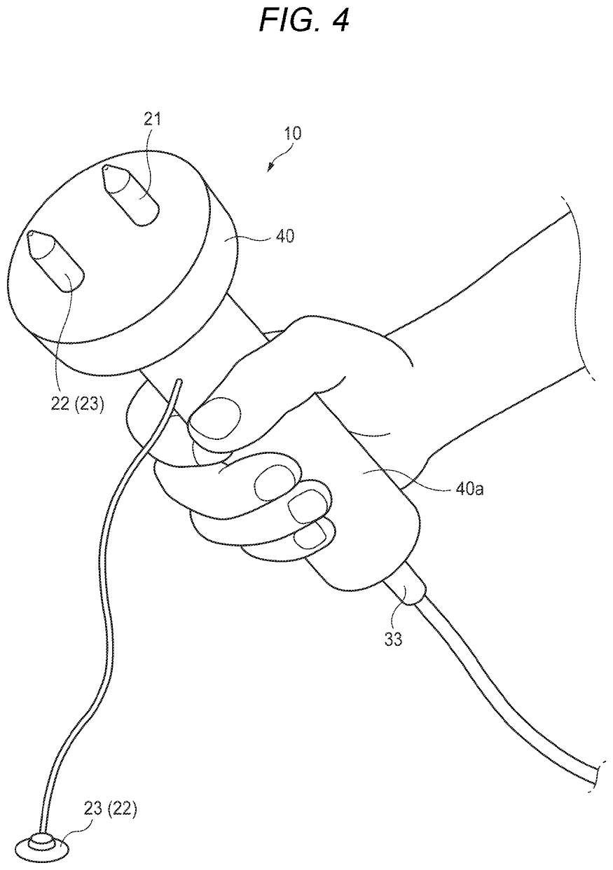

As illustrated in FIG. 4, one of the second electrode 22 and the third electrode 23 may be connected to the body 40 through a signal line. In the case where the first electrode 21 and the second electrode 22 are supported by the body 40, an electroencephalogram corresponding to the potential difference between the first electrode 21 and the second electrode 22 can be acquired while ensuring the degree of freedom in the attachment position of the third electrode 23. In the case where the first electrode 21 and the third electrode 23 are supported by the body 40, an electroencephalogram corresponding to the potential difference between the first electrode 21 and the second electrode 22 can be acquired while ensuring the degree of freedom in the attachment position of the second electrode 22.

The shapes of the first electrode 21, the second electrode 22, and the third electrode 23 may be adequately determined. In the embodiment illustrated in FIG. 3, all of the first electrode 21, the second electrode 22, and the third electrode 23 are protruded from the body 40.

According to the configuration, the measurement is hardly obstructed by the head hair of the subject. Therefore, it is possible to assist more rapid and easier acquisition of an electroencephalogram.

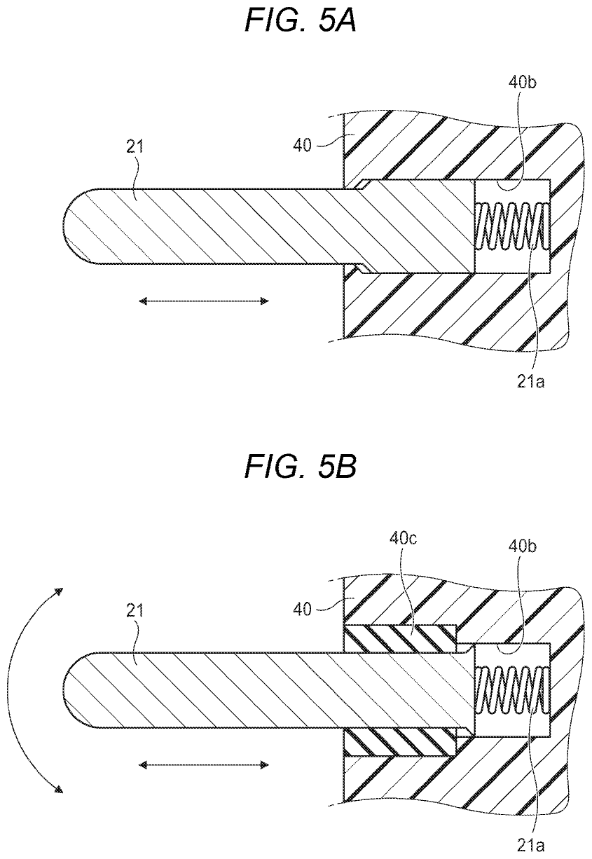

In this case, the first electrode 21 may be displaceable in the protrusion direction. As illustrated in FIG. 5A, for example, the first electrode 21 may be supported so as to be slidable in a hole 40b that is formed in the body 40. An electrically conductive spring 21a is housed in the hole 40b. The electrically conductive spring 21a is always contacted with the first electrode 21, and urges the first electrode 21 toward the tip end thereof. The first electrode 21 is electrically connected to the signal processing circuit 31 through the electrically conductive spring 21a.

When the first electrode 21 is pressed against the scalp of the subject, the first electrode 21 is slid in the hole 40b by the reaction force caused by the scalp, in the direction toward the basal end of the electrode, to compress the electrically conductive spring 21a. The first electrode 21 stays in a position where the elastic restoring force of the electrically conductive spring 21a balances with the reaction force caused by the scalp. This configuration can be applied also to at least one of the second electrode 22 and third electrode 23 that are supported by the body 40.

According to the configuration, when at least one of the first electrode 21, the second electrode 22 and the third electrode 23 is displaced in the protrusion direction, it is possible to cope with the difference in scalp shape among subjects. Therefore, it is possible to assist more rapid and easier acquisition of an electroencephalogram.

Moreover, the first electrode 21 may be displaceable in a direction intersecting with the protrusion direction. As illustrated in FIG. 5B, for example, an elastic sleeve 40c may be housed in the hole 40b. The first electrode 21 is supported by the body 40 while being slidable along the inner circumference of the sleeve 40c.

When the first electrode 21 is pressed against the scalp of the subject, the first electrode 21 receives the reaction force caused by the scalp. The reaction force may contain also a component in a direction intersecting with the protrusion direction of the first electrode 21. The first electrode 21 may be inclined from the initial position while the sleeve 40c is elastically deformed by the component. The first electrode 21 stays in a position where the elastic restoring force of the sleeve 40c balances with the reaction force caused by the scalp. This configuration can be applied also to at least one of the second electrode 22 and third electrode 23 that are supported by the body 40.

According to the configuration, when at least one of the first electrode 21, the second electrode 22, and the third electrode 23 is displaced (inclined) in a direction intersecting with the protrusion direction, it is possible to cope with the difference in scalp shape among subjects. Therefore, it, is possible to assist more rapid and easier acquisition of an electroencephalogram.

In the example illustrated in FIG. 5B, the configuration where the displacement of the first electrode 21 in the protrusion direction is allowed may be omitted.

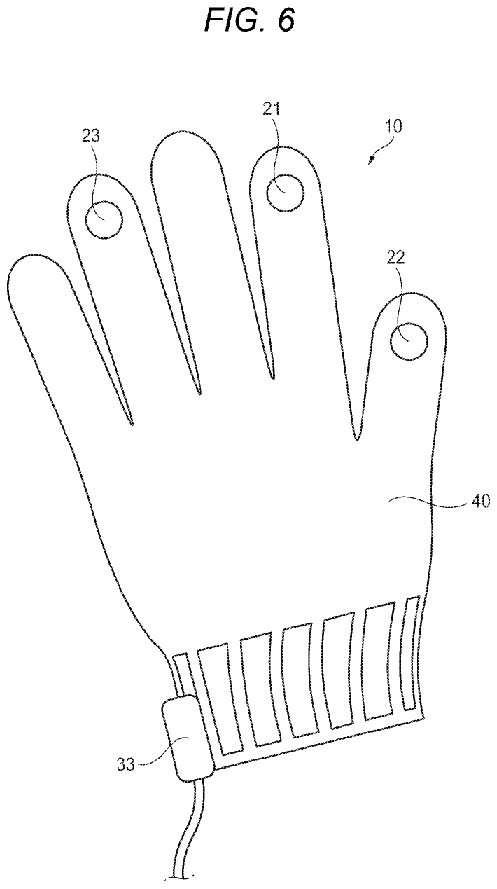

FIG. 6 illustrates a third embodiment of the electroencephalogram measuring apparatus 10. In the embodiment, the electroencephalogram measuring apparatus 10 exhibits a glove-like appearance. When the user inserts the hand into the body 40 having a glove-like shape, the user can hold the electroencephalogram measuring apparatus 10. In the specification, the term “is held by the hand of the user during measurement” has a meaning that includes also the above-described holding manner.

The first electrode 21 is supported on the tip end of a portion of the body 40 into which the index finger is to be inserted. The second electrode 22 is supported on the tip end of a portion of the body 40 into which the thumb is to be inserted. The third electrode 23 is supported on the tip end of a portion of the body 40 into which the ring finger is to be inserted.

The user presses through the index finger the first electrode 21 against a position which is in the head of the subject, and from which an electroencephalogram is to be measured. Also the second electrode 22 and the third electrode 23 are pressed against the head of the subject through the thumb and the ring finger, respectively. Therefore, an electroencephalogram of the subject corresponding to the potential difference between the first electrode 21 and the second electrode 22 is obtained. A signal indicating the electroencephalogram (the electroencephalogram signal S in FIG. 1) is output through the output interface 33.

According to the configuration, the user can acquire an electroencephalogram of the subject corresponding to the potential difference between the first electrode 21 and the second electrode 22, by performing a simple operation in which it is required only to press the own fingers against the head of the subject through the body 40 having a glove-like shape. Even during measurement, moreover, the position where the first electrode 21 is placed can be freely changed by moving the position of the index finger. Therefore, it is possible to assist more rapid and easier acquisition of an electroencephalogram.

The positions of the first electrode 21, the second electrode 22, and the third electrode 23 are not limited to the portions which are in the body 40, and in which the index finger, thumb, and ring finger of the user are to be inserted respectively, and may be adequately changed.

As in the embodiment illustrated in FIG. 4, alternatively, at least one of the second electrode 22 and the third electrode 23 may be connected to the body 40 through a signal line. In the case where the first electrode 21 and the second electrode 22 are supported by the body 40, an electroencephalogram of the subject corresponding to the potential difference between the first electrode 21 and the second electrode 22 can be acquired while suppressing the displacement of the third electrode 23 during measurement. In the case where the first electrode 21 and the third electrode 23 are supported by the body 40, an electroencephalogram of the subject corresponding to the potential difference between the first electrode 21 and the second electrode 22 can be acquired while suppressing the displacement of the second electrode 22 during measurement. In the case where only the first electrode 21 is supported by the body 40, an electroencephalogram of the subject corresponding to the potential difference between the first electrode 21 and the second electrode 22 can be acquired while suppressing the displacements of the second electrode 22 and the third electrode 23 during measurement.

In the above-described embodiments, as illustrated in FIG. 1, the electroencephalogram measuring apparatus 10 may include a notifying section 34. The notifying section 34 is configured so as to output a sound that corresponds to a change over time of the potential difference between the first potential VI and the second potential V2 (i.e., the electroencephalogram signal S). Specifically, the loudness (volume) of the output sound may be expressed by the amplitude of the change over time of the potential difference, and the level (pitch) of the output sound may be expressed by the frequency of the change over time of the potential difference. Alternatively, the notifying section may be configured so as to output a specific sound in the case where the electroencephalogram signal S indicating an abnormality of the brain is acquired.

According to the configuration, the user may be assisted to know the condition of the subject, by the sound that is output from the notifying section 34. Even in a situation where an environment in which an electroencephalogram can visually check is hardly ensured, therefore, it is possible to assist more rapid and easier acquisition of an electroencephalogram.

A skilled medical person can know the condition of the brain of the subject from a recording sound caused by a pen of an electroencephalograph. The term “a recording sound that is caused by a pen of an electroencephalograph” that is used in the specification means a frictional sound that, in the case where a pen records the waveform of an electroencephalogram on a recording sheet of an electroencephalograph, is produced between the pen tip and the recording sheet. Preferably, therefore, the sound output from the notifying section 34 is a sound imitating a recording sound that is caused by a pen of an electroencephalograph. Even in a situation where an environment in which an electroencephalogram can be visually checked is hardly ensured, consequently, the condition of the subject can be known more easily.

The notifying section 34 may be realized by a speaker that is built in the body 40, or in the form of an earphone or headphone that has a portion which is to be attached to the ear of the user. According to the configuration, the sound output from the notifying section 34 can be easily distinguished from ambient noise. Moreover, a situation where the sound output from the notifying section 34 spreads to the periphery, and is erroneously recognized as noise can be prevented from occurring.

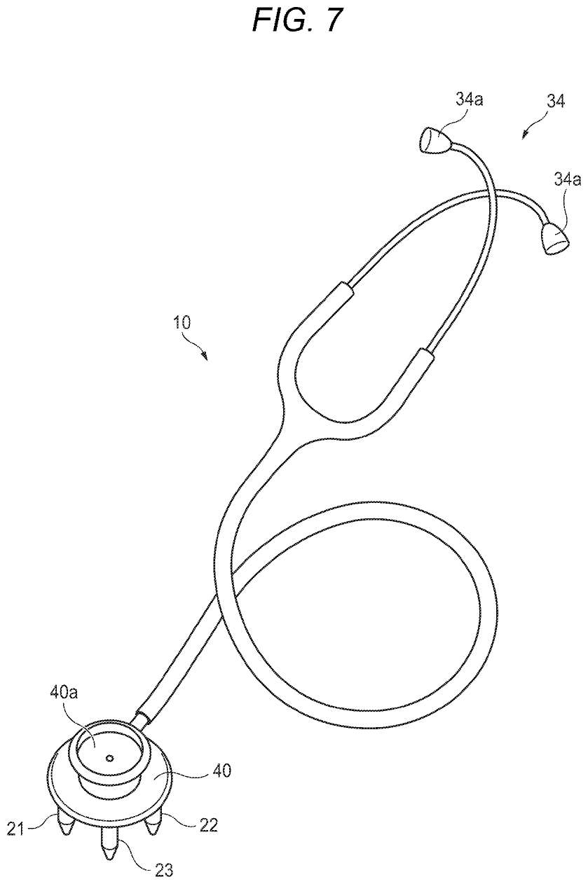

FIG. 7 illustrates a fourth embodiment of the electroencephalogram measuring apparatus 10 in which, as described above, the notifying section 34 has a portion that is to be attached to the ear of the user. In the embodiment, the electroencephalogram measuring apparatus 10 exhibits an appearance of a stethoscope-like shape. The body 40 having a shape that is similar to a chest piece of a stethoscope may include a bell-shaped holder 40a that is to be held by the hand of the user during measurement. The first electrode 21, the second electrode 22, and the third electrode 23 are supported on the side of the body 40 that is opposite to the holder 40a.

The user presses the first electrode 21 against a position which is in the head of the subject, and from which an electroencephalogram is to be measured. Also the second electrode 22 and the third electrode 23 are pressed against the head of the subject. Therefore, an electroencephalogram of the subject corresponding to the potential difference between the first electrode 21 and the second electrode 22 is acquired. A signal indicating the electroencephalogram (the electroencephalogram signal S in FIG. 1) is output through the output interface 33.

The notifying section 34 may include ear attaching portions 34a having a shape that is similar to an ear chip of a stethoscope. The ear attaching portions 34a are an example of the portion that is to be attached to the ear of the user. The user can listen to a sound corresponding to the electroencephalogram signal S, through the ear attaching portions 34a.

According to the configuration, the user can acquire an electroencephalogram corresponding to the potential difference between the first electrode 21 and the second electrode 22, by performing a simple operation in which it is required only to, while holding the holder 40a by the hand, press the first electrode 21, second electrode 22, and third electrode 23 that are held by the body 40, against the head of the subject. Even during measurement, moreover, the position where the first electrode 21 is placed can be freely changed with the user's hand that holds the holder 40a. Therefore, it is possible to assist more rapid and easier acquisition of an electroencephalogram.

As in the embodiment illustrated in FIG. 4, at least one of the second electrode 22 and the third electrode 23 may be connected to the body 40 through a signal line.

The above-described embodiments are mere examples for facilitating understanding of the present disclosure. The configurations of the embodiments may be adequately changed or improved without departing from the spirit of the present disclosure.

The electroencephalogram measuring apparatus 10 of each of the above-described embodiments includes the third electrode 23 which provides a potential that functions as the reference of the potential difference between the first electrode 21 and the second electrode 22. According to the configuration, influences that are exerted on the electroencephalogram measurement by in-phase noise are easily suppressed. However, the third electrode 23 may be omitted. Also in this case, an electroencephalogram of the subject corresponding to the potential difference between the first electrode 21 and the second electrode 22 can be acquired by performing a simple operation in which the second electrode 22 is attached to an appropriate position, and then the first electrode 21 is placed in an arbitrary position of the head of the subject. Even during measurement, moreover, the position where the first electrode 21 is placed can be freely changed with the user's hand that holds the body 40. Therefore, it is possible to assist more rapid and easier acquisition of an electroencephalogram that is requested particularly in a site such as emergency medicine.

According to an aspect of the present disclosure, there is provided an electroencephalogram measuring apparatus comprising: a first electrode that is to be placed in a first position of a head of a subject; a second electrode that is to be placed in a second position of the subject; and a body supporting at least the first electrode, and having a portion that is to be held by a hand of a user.

In an electroencephalogram measurement method according to the International 10-20 electrode placement system, the work of attaching many electroencephalogram electrodes to the all predetermined portions requires a long time. In a field where an abnormality of the brain of the patient must be rapidly recognized, such as an emergency medical care, it is requested to acquire an electroencephalogram more rapidly and easily.

According to the above-described configuration, an electroencephalogram of the subject corresponding to the potential difference between the first electrode and the second electrode can be acquired by performing a simple operation in which the second electrode is attached to an appropriate position, and then the first electrode is placed in the first position of the head of the subject. Even during measurement, moreover, the first position can be freely changed with the user's hand that holds the body. Therefore, it is possible to assist more rapid and easier acquisition of an electroencephalogram that is requested particularly in a site such as emergency medicine.

What is claimed is:

1. An electroencephalogram measuring apparatus comprising:
a first electrode that is to be placed in a first position of a head of a subject;
a second electrode that is to be placed in a second position of the subject; and
a body supporting at least the first electrode, and having a portion that is to be held by a hand of a user.

2. The electroencephalogram measuring apparatus according to claim 1, further comprising:
a third electrode which provides a potential that functions as a reference of a potential difference between the first electrode and the second electrode.

3. The electroencephalogram measuring apparatus according to claim 1, wherein
the body supports at least one of the second electrode and a third electrode.

4. The electroencephalogram measuring apparatus according to claim 3, wherein
the first electrode and at least one of the second electrode and the third electrode is protruded from the body.

5. The electroencephalogram measuring apparatus according to claim 4, wherein
at least one of the first electrode, the second electrode and the third electrode is supported by the body in a manner that the at least one electrode is displaceable in a protrusion direction.

6. The electroencephalogram measuring apparatus according to claim 4, wherein
at least one of the first electrode, the second electrode and the third electrode is supported by the body in a manner that the at least one electrode is displaceable in a direction intersecting with a protrusion direction.

7. The electroencephalogram measuring apparatus according to claim 1, further comprising: a notifying section configured to output a sound that corresponds to a change over time of a potential difference between the first electrode and the second electrode.

8. The electroencephalogram measuring apparatus according to claim 7, wherein
a loudness of the sound is expressed by an amplitude of the change over time, and a pitch of the sound is expressed by a frequency of the change over time.

9. The electroencephalogram measuring apparatus according to claim 7, wherein
the sound is a sound imitating a recording sound that is caused by a pen of an electroencephalograph.

10. The electroencephalogram measuring apparatus according to claim 7, wherein
the notifying section is configured to, in a case where the change over time of the potential difference corresponds to an abnormality of a brain, output a specific sound.

11. The electroencephalogram measuring apparatus according to claim 7, wherein
the notifying section has a portion that is to be attached to an ear of the user.

12. The electroencephalogram measuring apparatus according to claim 1, wherein the body has a portion that is to be held by the hand of the user during measurement.

13. The electroencephalogram measuring apparatus according to claim 1, wherein the portion that is to be held by the hand of the user has a pen-like shape.

14. The electroencephalogram measuring apparatus according to claim 1, wherein the portion that is to be held by the hand of the user has a rod-like shape.

15. The electroencephalogram measuring apparatus according to claim 1, wherein the portion that is to be held by the hand of the user has a bell-like shape.
